(19) United States
(12) Patent Application Publication
(10) Pub. No.: US 2010/0139666 A1
(43) Pub. Date: Jun. 10, 2010

(54) DEVICE INTENDED TO CAUSE A DISPLACEMENT IN THE MANDIBLE IN RELATION TO THE MAXILLA

(76) Inventor: Pierre Bonnaure, Rennes (FR)

Correspondence Address: NIXON & VANDERHYE, PC, 901 NORTH GLEBE ROAD, 11TH FLOOR, ARLINGTON, VA, 22203, US

(21) Appl. No.: 12/515,080

(22) PCT Filed: Nov. 21, 2007
(86) PCT No.: PCT/EP2007/010077

§ 371 Date: Nov. 24, 2009

(30) Foreign Application Priority Data
Nov. 23, 2006 (FR) 06/10284

(51) Int. Cl. A61F 5/56

(52) U.S. Cl. 128/848

(57) ABSTRACT

The present invention concerns a device intended to cause displacement of the mandible in relation to the maxilla, which is neither inconvenient for the user nor bulky, and which allows displacement of the mandible in relation to the upper maxilla in a postero-anterior direction and/or a transverse direction in relation to the plane of the maxilla and/or perpendicular in relation to the plane of the maxilla.

The device (D) according to the invention comprises at least one mandible displacement means (1) intended to be fixed to the upper maxilla (MX) and the mandible (MD) by two fixing means (6, 7) respectively and is characterised in that said mandible displacement means (1) comprises an upper part (11) intended to be linked to said upper maxilla (MX) and a lower part (12) intended to be linked to said mandible (MD), said upper part (11) and lower part (12) being intended to be linked together via a linking means (14), said linking means (14) comprising a means of adjustment (16) of the displacement of the mandible (MD) in relation to the upper maxilla (MX).

The present invention concerns a device intended to cause a displacement in the mandible in relation to the maxilla of a user.

Known devices intended to cause a displacement in the mandible are devices which provoke an advance of the mandible in relation to the maxilla and are otherwise known as orthoses. Such devices are used to treat obstructive respiratory disorders during sleep such as high resistance of the upper airway syndrome, snoring, apnoea and hyponoea.

Obstructive respiratory disorders during sleep are due to poor ventilation of the patient during sleep by obstruction of his airways. The respiratory tracts, in particular the pharynx, are obstructed for example in the case of too great an uvula, too thick a soft palate, the mandible being positioned behind the maxilla, or a poor position of the base of the tongue.

The consequences of obstructive respiratory disorders during sleep are numerous, such as for example drowsiness during the day, difficulty in concentration, depression or a large number of physiological problems.

In the child, obstructive respiratory disorders during sleep are also common. Their consequences are various problems such as problems of growth, hyperactivity, concentration or an absence of enlargement of the palate arch. One of the causes of these problems is poor growth of the mandible in the postero-anterior direction, leading to obstruction of the respiratory tracts. Solving the problem of mandible growth in the child allows avoidance of obstructive disorders during sleep in the adult.

Medicine has an effective treatment, but this is both expensive and complex: ventilation by continuous positive pressure (VPPC). In order to be correctly ventilated, the patient must wear during the night a mask providing insufflation through the nose. Unfortunately certain patients cannot tolerate the noise caused by the machine.

It is also possible to use surgery, for example to cut the soft palate when it is too thick, or to move the chin forward.

Another less intrusive solution with fewer risks is to use a mandible advance orthosis. The principle is to move the mandible forward by a few millimetres in relation to its original position. This advance clears the pharynx and allows correct ventilation of the patient.

The best known mandible advance orthosis is certainly Herbst's rod. To fit this orthosis, the upper and lower rows of teeth are each covered by a rigid channel made of thermoformed acrylic resin and well known to the person skilled in the art. Two metal rods are arranged on either side of the upper and lower dental arches on their outer faces, to link the upper and lower rows of teeth. The two rods are attached to the channel covering the upper teeth by anchoring means arranged at the level of the last two molars respectively. The two rods are attached to the channel covering the row of lower teeth by anchoring means arranged at the two canines respectively.

Also known are orthoses comprising two thermoformed channels covering the upper and lower rows of teeth respectively and also comprising two elastic elements fixed into hooks linking the two channels laterally. The lower jaw is thus held in an advanced position.

These mandible advance devices have the drawback of sometimes being very bulky. For some patients, such as those using a Herbst rod, the rigidity can cause inconvenience for the user. Also no known device has the possibility of fine adjustment and these devices are therefore restricted to pushing the mandible in the postero-anterior direction in relation to the upper maxilla, and do not allow the mandible to be also drawn in a direction both parallel to the plane formed by the upper maxilla and perpendicular to the postero-anterior direction.

The present invention concerns a mandible advance device which does not have the said drawbacks. In particular the object of the invention is to propose a device which causes no inconvenience for the user, is not bulky and allows displacement of the mandible in relation to the upper maxilla in a postero-anterior direction and/or a transverse direction in relation to the plane of the maxilla and/or perpendicular in relation to the plane of the maxilla.

In the present invention, a direction described as transverse in relation to the plane of the maxilla is a direction which is both parallel to the plane formed by the upper maxilla and perpendicular to the postero-anterior direction.

To this end a device according to the invention intended to cause displacement of the mandible in relation to the upper maxilla when worn by a user, said mandible and said maxilla comprising a lower dental arch and an upper dental arch respectively, comprises at least one mandible displacement means intended to be fixed to the upper maxilla and the mandible by two fixing means respectively, and is characterised in that said mandible displacement means comprises an upper part intended to be linked to the upper maxilla and a lower part intended to be linked to the mandible, said upper and lower parts being intended to be linked to each other via a linking means, said linking means comprising a means of adjustment of the displacement of the mandible in relation to the upper maxilla.

The device according to the invention also has the advantage of comprising a means of adjustment of the displacement of the mandible in relation to the upper maxilla both in a postero-anterior direction in relation to the plane of the upper maxilla and a direction transverse in relation to this plane. In fact each mandible displacement means comprises its own means of adjustment of mandible displacement, which allows adjustment of the displacement of the mandible in the postero-anterior direction. If the device comprises at least two mandible displacement means, different settings of each adjustment means will cause a displacement of the mandible in the postero-anterior direction and in the transverse direction in relation to the plane of the upper maxilla.

The means of adjustment of the mandible displacement also has the advantage of being able to control this mandible displacement in relation to the maxilla progressively from one period to another.

Furthermore the structure of each mandible displacement means in two parts, i.e. a lower part and an upper part which can be linked together by a linking means, has the advantage of facilitating use of the device according to the invention as the two parts can be joined before or after being placed in the mouth.

According to a preferred embodiment of the invention, said linking means and said adjustment means are one and the same means.

According a preferred embodiment of the invention, said adjustment means comprises a tap-threaded tube forming said lower part and a thread forming said upper part, or vice versa, said thread being intended to be screwed into said tap-threaded tube.

Thus the link between the lower part and the upper part and the adjustment of the displacement of the mandible in relation to the maxilla are achieved by screwing the thread into the tap-threaded tube.

According to another embodiment, said adjustment means comprises at least one hook forming said lower part and at least one notch forming said upper part, or vice versa, said hook being intended to engage in said notch.

Thus the link between the lower part and the upper part and the adjustment of the displacement of the mandible in relation to the maxilla are achieved by hooking, i.e. by progression of a hook from one notch to the next.

Advantageously said device comprises a blocking means for said adjustment means.

In one embodiment of the invention, said blocking means is a bolt screwed to said thread.

Once the displacement of the mandible in relation to the maxilla has been set by the adjustment means, it can be advantageous to block the adjustment. The blocking means allows the position of the mandible to be blocked in relation to the maxilla. For example when the adjustment means comprises a thread screwed into a tap-threaded tube, it prevents the tap-threaded tube from turning around the thread, or in other words prevents the thread from not screwing in or becoming unscrewed.

According to a characteristic of the invention, the lower part of said mandible displacement means comprises a flexible rod. This characteristic allows great freedom of movement when the device is worn by the user and hence great comfort.

According to one embodiment of the invention, the means for fixing said mandible displacement means to the upper maxilla is arranged at the canine and/or premolars of said upper dental arch, and said means for fixing said mandible displacement means to the mandible is arranged at the molars of said lower dental arch when said device is worn by the user.

When the device is worn by the user, the mandible displacement means is therefore not perpendicular to the plane formed by the maxilla. This allows displacement of the mandible in the postero-anterior direction in relation to the maxilla.

According to one embodiment of the invention, said means of fixing said mandible displacement means to the upper maxilla comprises a tube intended to house one end of said upper part of said mandible displacement means, said tube being intended to be fixed on said upper dental arch or said upper maxilla directly or via a linking means.

According to another embodiment of the invention, said means of fixing said mandible displacement means to the upper maxilla comprises a ring forming said upper part of said mandible displacement means and a hook attached to said upper maxilla directly or via a linking means, said ring being intended to be hooked onto said hook.

According to one embodiment of the invention, said means of fixing said mandible displacement means to the mandible comprises a ring forming said lower part of said mandible displacement means and a hook attached to said mandible directly or via a linking means, said ring being intended to be hooked onto said hook.

In a preferred embodiment of the invention said means of fixing said mandible displacement means to the mandible comprises a means of linking to said mandible in which one end of said lower part of said mandible displacement means is cast.

Advantageously said linking means is selected from a thermoformed channel, a dental implant, a dental crown, and orthodontic ring, at least one micro-implant or at least one mini-screw screwed into the gum or below the gum.

The thermoformed channel, the dental implant, the dental crown or orthodontic ring are arranged on one dental arch. The micro-implant(s) or mini-screw(s) are screwed into the bone through the gum or below the gum. The latter can for example be used by a user of a device according to the invention whose upper maxilla and/or mandible have no upper and/or lower dental arch respectively.

In another preferred embodiment of the invention, said device comprises both a thermoformed channel intended to cover at least part of said lower dental arch and a thermoformed channel intended to cover at least part of said upper dental arch.

According to a characteristic of the invention, the upper part of said mandible displacement means comprises a U-shaped rod, one arm of which is intended to be fixed to said upper maxilla by said means of fixing said mandible displacement means to said upper maxilla.

The device according to the invention can also comprise at least one wedge intended to be fixed to one of said upper or lower dental arches directly or via a linking means. Said wedge allows the user's mouth to be held more or less open when the device is worn by the user.

Said linking means is selected for example from a thermoformed channel, a dental implant, a dental crown or an orthodontic ring.

The device according to the invention can also comprise at least one elastic section intended to be fixed to said upper maxilla and said mandible by two hook means respectively attached to said upper maxilla and said mandible directly or via two linking means respectively, such as said linking means described above.

Said elastic element allows the user's mouth to be held more or less closed when the device is worn by the user.

Said wedge and said elastic element thus allow displacement of the mandible in relation to the maxilla in a direction perpendicular to the plane of the maxilla.

The above-mentioned characteristics of the invention and others will become clearer when reading the following description of an embodiment, said description being produced in relation to the attached drawings in which:

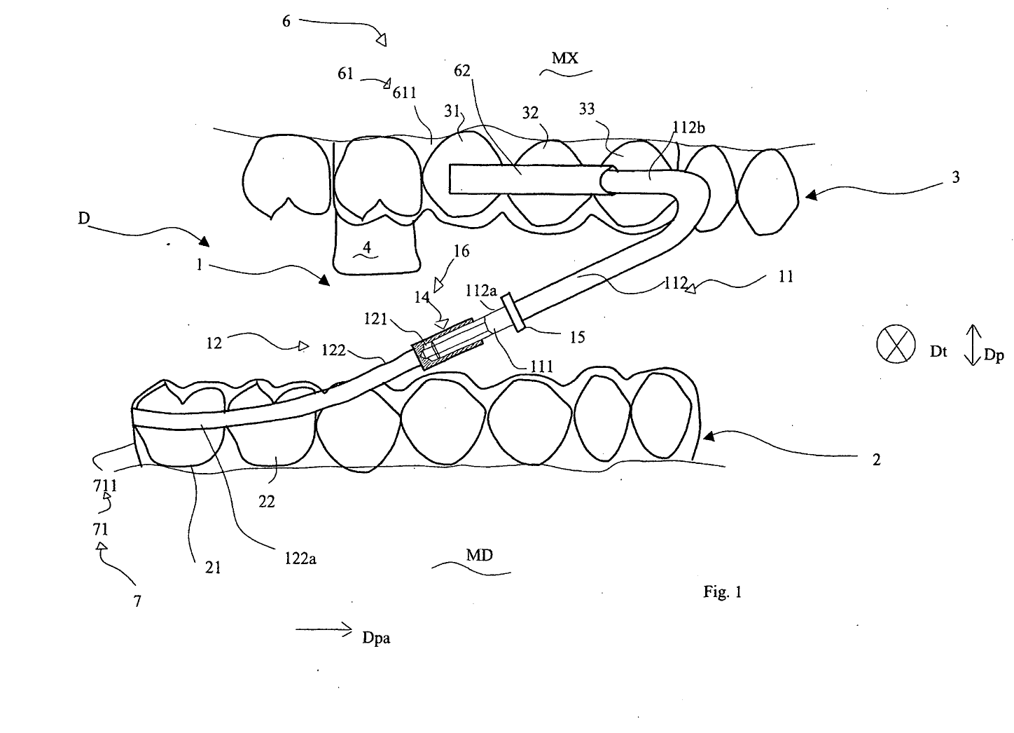

FIG. 1 shows a device intended to cause displacement of the mandible in relation to the upper maxilla of a user, in a preferred embodiment of the invention;

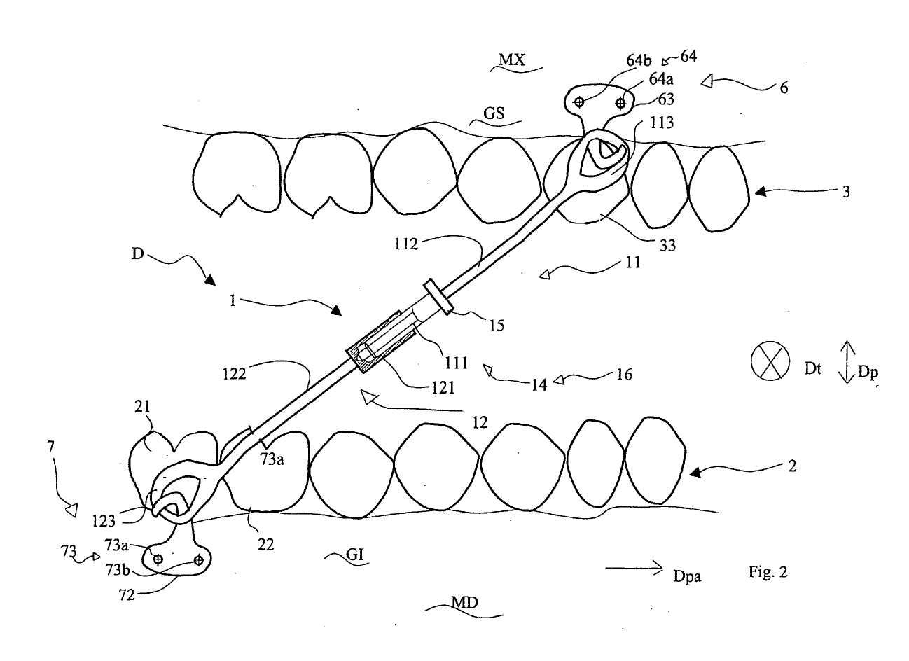

FIG. 2 shows a device intended to cause displacement of the mandible in relation to the upper maxilla of a user, in another embodiment of the invention; and

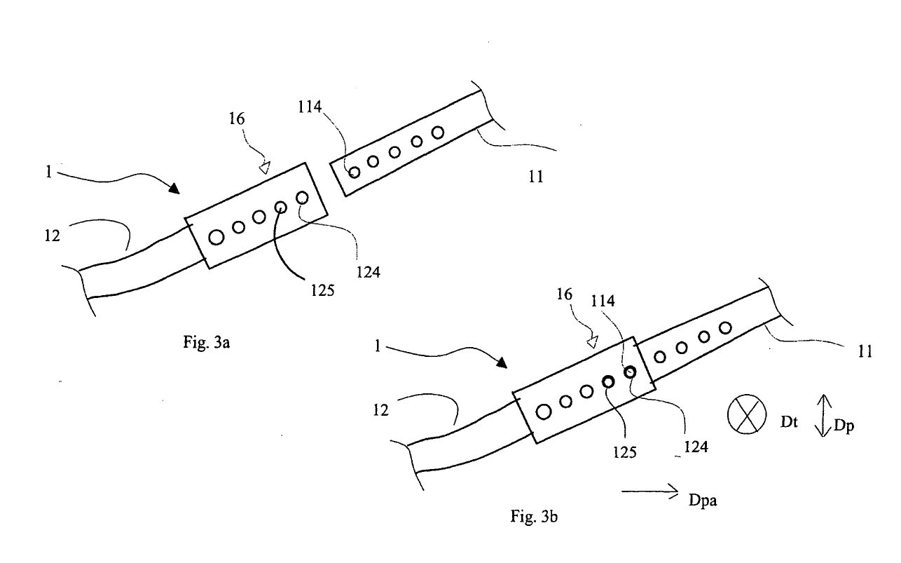

FIGS. 3a and 3b show a means of adjustment of the mandible displacement in another embodiment of the invention.

FIG. 1 shows the upper maxilla MX comprising the upper dental arch 3 and the mandible MD comprising the lower dental arch 2 of a user of device D according to the invention.

Device D is intended to allow displacement of the mandible MD in relation to the upper maxilla MX in a postero-anterior direction Dpa and/or a transverse direction Dt in relation to the plane of the maxilla MX and/or a perpendicular direction Dp in relation to the plane of the upper maxilla MX.

The device D comprises a mandible displacement means 1 comprising a lower part 12 and an upper part 11 which are linked together via a linking means 14.

The linking means 14 comprises a thread 111 forming the upper part 11 and a tap-threaded tube 121 forming the lower part 12. The thread 111 is screwed into the tap-threaded tube 121 so as to link the upper part 11 to the lower part 12.

The upper part 11 comprises a U-shaped rod 112. An arm 112a of said rod 112 contains the thread 111. Another branch 112b of rod 112 is housed inside a tube 62 which is fixed to the upper arch 3 via a linking means 61. The linking means 61 is for example a thermoformed channel 611 well known to the person skilled in the art, and which covers part of the dental arch 3 comprising at least premolars 31, 32 and canine 33. The tube 62 and the linking means 61 form a means 6 for fixing the mandible displacement means 1 to the upper maxilla MX. The fixing means 6 is arranged at the premolars 31, 32 and the canine 33.

The thermoformed channel 611 can be removable or cemented, for example glued, to part or the whole of the dental arch 3.

The lower part 12 comprises a rod 122, advantageously made of flexible material so as to allow voluntary movement of the mandible MD.

The lower part 12 of the mandible displacement means 1 is fixed to the mandible MD by a fixing means 7.

The fixing means 7 comprises a means 71 for linking the device 1 to the mandible MD, in which an end 122a of the lower part 12 is cast. This linking means 71 is a thermoformed channel 711 which covers all or part of the lower dental arch 2 and may be removable.

The end 122a of the lower part 12 is cast at the molars 21, 22. The mandible displacement means 1 is therefore not perpendicular to the plane formed by the maxilla MX.

The thermoformed channel 711 for example covers the whole of the dental arch 2 and is removable.

The linking means 14 in this embodiment is also a means 16 for adjustment of the displacement of the mandible MD in relation to the maxilla MX. In fact screwing the thread 111 into the tap-threaded tube 121 causes displacement of the mandible MD in relation to the maxilla MX in a postero-anterior direction Dpa. The progression of the thread 111 in the threaded tube 121 can thus be achieved from one period to the next, for example from one day to the next.

In a preferred embodiment of the invention, the device D comprises a second mandible displacement means (not shown) intended to be arranged symmetrically in relation to a central longitudinal axis parallel to the plane of the upper maxilla MX, for example on each side of dental arches 2 and 3.

It is then possible to adjust the displacement of the mandible MD in relation to the upper maxilla MX both in a transverse direction Dt in relation to the plane of the upper maxilla MX and in a postero-anterior direction Dpa in relation to the maxilla MX. In fact the screwing of the threads 111 into the tap-threaded tubes 121 of the two displacement means 1 respectively of device D may differ, which causes a displacement of the mandible in a transverse direction Dt in relation to the plane of the maxilla MX in addition to the displacement in the postero-anterior direction Dpa.

Device D as shown on FIG. 1 may again comprise a blocking means 15 of the adjustment means 16. Said blocking means 15 can for example comprise a nut screwed onto the thread 111 so as to prevent any rotation of the tap-threaded tube 121 around the thread 111.

The device D can also comprise a wedge 4 fixed, for example glued, to the dental arch 3 via the linking means 61 for example of the thermoformed channel. The wedge 4 prevents contact between the upper and lower dental arches 3 and 2 respectively. Thus the wedge 4 allows displacement of the mandible MD in relation to the maxilla MX in a perpendicular direction Dp in relation to the plane of maxilla MX.

In a variant of the invention (not shown), the device can comprise an elastic element intended to be fixed to the upper maxilla MX and the mandible MD via two linking means respectively, such as the linking means 61 and 71 described above. The elastic element allows the upper and lower dental arches 3 and 2 respectively to be held against each other. The elastic element also allows displacement of the mandible MD in relation to the maxilla MX in a perpendicular direction Dp in relation to the plane of the maxilla MX.

FIG. 2 shows another embodiment of device D according to the invention.

The device D comprises a mandible displacement means 1 comprising an upper part 11 and a lower part 12.

The lower part 12 comprises a flexible rod 122, one end of which is shaped as a ring 123. Also the upper part 11 comprises a rod 112, one end of which is shaped as a ring 113.

The device D comprises a linking means 14 which is also an adjustment means 16 identical to that of the embodiment described above.

The lower part 12 of the mandible displacement means 1 is fixed to the mandible MD by a fixing means 7. The fixing means 7 is composed of the ring 123 of the lower part 12 and a hook 72 fixed to the mandible via a linking means 73. The linking means 73 comprises at least one micro-implant, for example two micro-implants 73a and 73b which are screwed into or under the lower gum GI below the molar 21 or between the molars 21 and 22. The fixing means 7 is therefore arranged at the user's molars.

The upper part 11 of the mandible displacement means 1 comprises a rod 112, one end of which is shaped as a ring 123.

The upper part 11 is fixed to the maxilla MX via fixing means 6. The fixing means 6 comprises the ring 113 comprising the upper part 11 and a hook 63 attached to the maxilla MX via a linking means 64. The linking means 64 comprises at least one micro-implant, for example two micro-implants 64a and 64b which are screwed into or under the upper gum GS above the canine 33.

The fixing means 6 is therefore arranged at the user's canine.

In another variant of the invention shown on FIGS. 3a and 3b, the upper part 11 of the mandible displacement means 1 comprises hooks such as hook 114, and the lower part 12 comprises notches such as notches 124 (FIG. 3a). The hooks such as hook 114 and the notches such as notch 124 constitute the means 16 for adjustment of the displacement of the mandible in relation to the maxilla. The person skilled in the art will understand that the hooks, such as hook 114, can conversely be located in the lower part 12 if the notches, such as notch 124, are in the upper part 11. The hooks, such as hook 114, are intended to engage in the notches, such as notch 124.

In this variant of the invention, the adjustment means 16 and the linking means 14 of the lower part 12 and the upper part 11 are the same means. In fact when the hook 114 is engaged in the notch 124, the link between the lower part 12 and the upper part 11 is established.

In this variant of the invention, the displacement of the mandible in relation to the maxilla in the postero-anterior direction Dpa is adjusted by hooking, i.e. by progression of hook 114 from one notch 124 to another notch 125 (FIG. 3b).

As in the first embodiment described above, the device D can comprise a second mandible displacement means (not shown) comprising the adjustment means 16 shown on FIGS. 3a and 3b. It is then possible to adjust the displacement of the mandible MD in relation to the upper maxilla MX both in a transverse direction Dt in relation to the plane of the upper maxilla MX and in a postero-anterior direction Dpa in relation to the maxilla MX. In fact the progression of the hooks, such as hook 114, in the notches, such as notches 124 or 125, of the two displacement means 1 may be different, which causes a displacement of the mandible in a transverse direction Dt in relation to the plane of the maxilla MX as well as the displacement in the postero-anterior direction Dpa.

The device according to the invention thus allows adjustment of the displacement of the mandible in relation to the maxilla of a user in several directions. The applications of such a device according to the invention are numerous. For example the device can be used in the adult to counter obstructive sleep disorders such as high resistance of the upper airways syndrome, apnoea and snoring. In the child the device can be used to promote mandible growth. In particular the device can be used to promote mandible growth at the same time as treating any other problem of orthodontics or jaws, for example together with a cleft palate.

1. Device intended to cause a displacement of the mandible in relation to the upper maxilla when worn by a user, said mandible and said maxilla being able to comprise a lower dental arch and an upper dental arch respectively, said device comprising at least one mandible displacement means, an upper part of which is intended to be linked to said upper maxilla via a fixing means, and a lower part is intended to be linked to said mandible by a fixing means comprising a channel, for example thermoformed, said upper part and lower part being intended to be linked to each other via a linking means, characterised in that said upper part comprises a U-shaped rod, one arm of which is intended to be housed inside a tube forming the means of fixing device to said maxilla, and in that said lower part comprises a flexible rod, one end of which is cast into the means of fixing said device to the mandible.

2. Device according to claim 1, characterised in that said fixing means comprises a channel, for example thermoformed, a dental implant, a dental crown, an orthodontic ring, at least one micro-implant or at least one mini-screw.

3. Device according to claim 1, characterised in that said means for fixing said mandible displacement means to the upper maxilla is arranged at the level of the canine and/or the premolars of the upper dental arch, and said means for fixing said mandible displacement means to the mandible is arranged at the level of one or more molars of the lower dental arch when said device is worn by the user.

4. Device according to claim 1, characterised in that said linking means comprises a means of adjustment of the displacement of the mandible in relation to the upper maxilla, comprising a thread forming said upper part and a tap-threaded tube forming the lower part or vice versa, said thread being screwed to said tap-threaded tube.

5. Device according to claim 1 characterised in that it also comprises at least one wedge intended to be fixed to one of said upper or lower dental arches directly or via a linking means.

6. Device according to any of the preceding claims, characterised in that it also comprises at least one elastic element intended to be fixed to said upper maxilla and said mandible by two hooking means respectively, attached to said upper maxilla and said mandible directly or to said linking means.
